(19) United States
(12) Patent Application Publication
(10) Pub. No.: US 2026/0355465 A1
(43) Pub. Date: Nov. 12, 2026

(54) OPTIMIZED DESIGN OF ROLLING MEMBRANE AS GUIDE EXTENSION CATHETER

(71) Applicant: BIOTRONIK AG, Buelach (CH)

(72) Inventors: Matthias Wesselmann, Ruedlinen (CH); Nadine Troes, Buelach (CH); Azadeh Mehrabi, Meilen (CH)

(21) Appl. No.: 19/125,573

(22) PCT Filed: Dec. 4, 2023
(86) PCT No.: PCT/EP2023/084114

§ 371 Date: Apr. 29, 2025

(30) Foreign Application Priority Data
Dec. 12, 2022 (EP) 22212728.4

(51) Int. Cl. A61M 25/01; A61M 25/00

(52) U.S. Cl. CPC A61M 25/0119; A61M 2025/0004; A61M 2025/0079

(57) ABSTRACT

A guide extension catheter includes a rolling membrane and an inner shaft. A first portion of the rolling membrane is connected to the inner shaft. A connection element is configured to temporarily connect a second portion of the rolling membrane to an outer shaft.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/084114, which was filed Dec. 4, 2023, which application claimed priority from EP Application 22212728.4, which was filed Dec. 12, 2022.

### FIELD OF THE INVENTION

A field of the invention concerns guide extension catheter devices.

### BACKGROUND

Changing life circumstances (e.g., stressful daily routines, unhealthy nutrition habits, lack of exercises, smoking, etc.) have led to an increase of coronary and/or artery related diseases over the course of the past years. These diseases can often lead to hazardous effects on a health state of a patient (e.g., a stenosis and/or occlusions of a blood vessel) and can in some cases deteriorate leading to an increased mortality among affected patients.

To avoid life-threatening circumstances, it can be crucial to diagnose respective diseases during an early stage of the disease and/or to allow a curing of the disease wherein the risk for the patient shall preferably be as low as possible.

A preferred minimally invasive method of supporting an early diagnosis and/or a curing of such diseases is based on the usage of catheters. A catheter can in most cases be implemented as a tubular-shaped, hose-like device which can be inserted into a body of a patient and can be moved along an artery and/or a vein up to the prevalent anomaly in the blood vessel of a patient (e.g., a stenosis).

Such catheters can be adapted to deliver a medical device (e.g., a balloon, a stent, etc.) and/or a drug to an affected region of a blood vessel of a patient to diagnose and/or cure the respective disease.

In some cases, the forward movement (e.g., in a distal direction) of a catheter and/or the backward movement (e.g., in a proximal direction) of a catheter can be rendered difficult especially in cases in which a blood vessel is occluded (e.g., calcified) and/or tortuous.

By way of example, in complex percutaneous coronary interventions (PCI), one of the biggest challenges is the inability to cross lesions by catheters and/or medical devices and/or medical drugs to the target lesions. In such cases, the buddy wire and/or anchoring balloon technique can be helpful to successfully complete a coronary intervention.

The mother-in-child technique is another powerful method for delivering a medical device to a certain target lesion. With a view to the mother-in-child technique, inserting a 4-French (Fr) catheter (child catheter) into a 6-Fr guiding catheter (mother catheter) can greatly improve the success rate for crossing lesions in which standard methods can fail. Therefore, also the “4-in-6” system is becoming a viable alternative for highly calcified, angular, or tortuous lesions.

Further in this regard, the rapid-exchange guide extension catheter (GEC) or guide catheter extension has been evolving. Previous studies have proposed a mechanism of its efficiency as increasing back-up support by deep intubation, as a conduit to decrease friction between the vessel wall and the catheter, or for improving co-axial alignment between the catheter and the lesion. It also facilitates stent and balloon delivery in complex coronary lesion, in complex anatomy and or in the left internal thoracic artery graft.

For percutaneous transluminal coronary angioplasty (PTCA) a guiding catheter is typically inserted into the access sheath together with the guidewire, once the guiding catheter is in place, the guide wire is pushed out of the guiding catheter and inside the coronaries and through the stenosis and then the guide extension catheter is introduced into the guiding catheter and find its way into the vessel by following the guide wire.

A guide extension catheter can be a monorail system. The guide extension portion in average has a length of 25 cm and it can be passed in a rapid exchange fashion and can extend beyond the distal end of the guide catheter. The guide extension catheter can be thinner than the guiding catheter by, e.g., 1 Fr and it can be designed to minimize trauma to the coronary artery. The proximal end of the guide extension catheter can be attached to a thin stainless-steel pushrod, which can be used to push and pull the guide extension catheter independent of the guiding catheter.

However, even modern guide extension catheters still have difficulties crossing calcified arteries, in particular, when 180° bends are present, due to excessive friction forces. Physicians can then use a balloon catheter to help the guide extension catheter to cross such a bend. This procedure requires placing the balloon distally from the guide extension catheter followed by an inflation of the balloon and a subsequent pushing of the guiding catheter over the balloon catheter while deflating the balloon catheter. However, in some cases, the balloon catheter and the guiding catheter can move relative to the vessel wall and can cause injuries to the intima due to excessive shear.

To summarize, if strongly bended blood vessels need to be investigated by a catheter, existing catheters can be limited in their applications and can cause undue risks for a health state of a patient and can unduly prolong a catheter-based intervention.

### SUMMARY OF THE INVENTION

A guide extension catheter of a preferred embodiment includes a rolling membrane and an inner shaft. A first portion of the rolling membrane is connected to the inner shaft. A connection element is configured to temporarily connect a second portion of the rolling membrane to an outer shaft.

### BRIEF DESCRIPTION OF THE DRAWINGS

The catheter can further include an external shaft at least partially enclosing the rolling membrane, wherein at least a proximal portion of the external shaft remains outside of the body of the patient during use of the catheter with the patient.

The following figures are provided to support the understanding of the present invention:

FIGS. 1A-1B: Illustration of an exemplary rolling membrane catheter without inner shaft;

FIGS. 2A-2C: Illustration of an exemplary rolling membrane catheter with an inner shaft and an additional connection element;

FIGS. 3A-3B: Illustration of an exemplary rolling membrane catheter with an inner shaft and an additional connection element;

FIG. 4: Illustration of exemplary components of a rolling membrane guide extension catheter;

FIG. 5: Illustration of an exemplary combination of an outer shaft with a rolling membrane catheter;

FIGS. 6A-6B: Illustration of an assembled rolling membrane guide extension catheter.

### DETAILED DESCRIPTION OF THE PREFERRED EMBODIMENTS

A (rolling membrane) guide extension catheter according to the invention includes a rolling membrane and an outer shaft (e.g. a guiding catheter).

According to first aspect of the invention the guide extension catheter includes or consists of a rolling membrane; an outer shaft; an inner shaft, which is at least partially arranged or arrangeable within the outer shaft; and wherein the rolling membrane is connected to the inner shaft, and optionally a connection element for (at least temporarily) connecting the rolling membrane to the outer shaft.

A first portion of the rolling membrane is connected to the inner shaft and the catheter further includes a connection element for temporarily (e.g. releasably) connecting a second portion of the rolling membrane to the outer shaft.

The rolling membrane can be adapted to be rolled out along a distal direction. The rolling membrane can be adapted to be rolled in along a proximal direction. The rolling membrane can be configured such that the rolling membrane extends to a larger extent in the longitudinal direction than it extends along a radial direction (i.e. a direction perpendicular to a longitudinal direction along which the rolling membrane is rolled out).

In some embodiments, the first portion of the rolling membrane can be a distal end of the rolling membrane as seen if the rolling membrane is fully rolled out. Thus, the rolling membrane can be configured to exit the outer shaft in the distal direction. Additionally or alternatively, the second portion of the rolling membrane can be a proximal end portion of the rolling membrane as seen if the rolling membrane is fully rolled in.

In some preferred embodiments, the outer shaft can be a guiding catheter.

The connection element is connected to a proximal portion of the rolling membrane and/or the connection element is adapted such that the connection element is connectable to the outer shaft.

By providing the catheter with a connection element for temporarily connecting a proximal portion of the rolling membrane to an outer shaft, a situational and non-permanent extension of the catheter can be facilitated. This can advantageously increase the field of possible applications, the catheter can be used for. For example, if a catheter can not be able to cross lesions in a blood vessel of a patient, the catheter can additionally be supplied with a suitable extension (e.g., by the outer shaft and the connection element to connect a rolling membrane) such that the lesion can be crossed without injuring an inner wall of a respective blood vessel of a patient. In combination with the rolling membrane, which can be adapted to safely and atraumatically propagate along a blood vessel of a patient, a situationally applicable guide extension catheter can be facilitated with a connected rolling membrane that can be released if not needed. For example, in cases in which the membrane can not be fully rolled out, it can be useful to implement the (releasable) connection at that (proximal) end of the membrane (which would be distal if the membrane were fully rolled out).

The connection element can be adapted for being inserted into the outer shaft and for establishing a connection between the rolling membrane and the outer shaft. The connection element can be connected to the rolling membrane by clamping. In some examples, also welding, gluing, etc. can be used. The connection between the rolling membrane and the outer shaft can be liquid and/or fluid tight.

The connection element and the rolling membrane can be made from a same material (combination). Alternatively, the connection element and the rolling membrane can differ in at least one constituent material.

The connection between the connection element and the rolling membrane can be temporary. However, in some examples, the connection between the connection element and the rolling membrane can be permanent.

The connection element can include a first diameter at a proximal portion of the connection element which can be equal or smaller than an inner diameter of the outer shaft and a second diameter at a distal portion of the connection element which can be smaller than the first diameter at the proximal portion of the connection element. This can enable a force fit of the connection element with the outer shaft.

If the first diameter of the proximal portion of the connection element is adapted to be smaller than an inner diameter of the outer shaft, convenient insertion and sliding properties of the connection element into and in the outer shaft can be facilitated. This can contribute to a simplified and overall convenient usability of the catheter.

Alternatively, the first diameter can be adapted to be larger than an inner diameter of the outer shaft. At least the proximal end portion of the connection element can be adapted to be flexible such that the proximal end portion of the connection element can be slightly squeezed prior to inserting the connection element into an inner lumen of the outer shaft. Since at least the proximal end portion of the connection element can try to relax back to its original diameter, a pushing force can be applied to an inner wall of the outer shaft which can be accompanied by friction/shear forces such that the connection element can be locked in the outer shaft, preferably by clamping.

By adapting the distal portion of the connection element with a diameter which is smaller than a diameter of a respective proximal portion, a convenient insertion of the connection element into the outer shaft can be facilitated.

The connection element can be connectable to a distal portion of the rolling membrane.

The connection element, preferably a proximal (end) portion of the connection element, can be connected to a distal (end) portion of the rolling membrane. The proximal (end) portion of rolling membrane can be connected to an inner shaft. Additionally or alternatively, a distal end portion of the connection element can be connectable to the rolling membrane. The connection element can generally be provided with an inner lumen. By adapting the connection element such that it is connectable to the distal portion of the rolling membrane, preferably at a proximal end portion of the connection element, at least a partial accommodation of the rolling membrane in the inner lumen of the connection element can be facilitated. This can contribute to a protection of the rolling membrane if the catheter needs to be applied with a certain pushing force along the distal direction to pass (minor) occlusions without damaging the rolling membrane.

The connection element can include an annular element for exerting a radially acting force onto a distal portion of the rolling membrane and an outer side of the outer shaft or an inner side of the outer shaft.

The annular element can be provided as a ring. The annular element can include a slid. The annular element can include a diameter that it is larger than the inner diameter of the outer shaft. By squeezing the annular element in a radial direction, the diameter of the annular element can be reduceable.

By inserting the annular element in the inner lumen of the outer shaft and placing a portion of the rolling membrane between the ring and the inner side of the outer shaft, the ring can exhibit the radially acting force onto the rolling membrane and the inner side of the outer shaft such locking the rolling membrane in the outer shaft.

The connection element can be adapted for being put over the outer shaft and for establishing a clamping connection between the rolling membrane and the outer shaft.

The annular element can include a diameter that is smaller than an outer diameter of the outer shaft. As a result, the annular element can tend to decrease its diameter when put over the outer shaft and can thus exhibit the radial force onto the outer side of the outer shaft which can advantageously be used for clamping the rolling membrane to the outer side of the outer shaft.

By adapting the connection element such that it can be put over the outer shaft, the rolling membrane can be clamped onto the outer side of the outer shaft. By such a configuration, an insertion of medical equipment (as it will further be described below) from the inner lumen of the outer shaft into the rolling membrane can be supported.

The connection element can include a hydrophilic coating and/or a swellable coating such as to swell if exposed to a liquid. If the connection element is inserted into the inner lumen of the outer shaft, the coating can be located in between the connection element and the inner side of the outer shaft.

The hydrophilic coating can be a hydrogel (e.g., polyvinylpyrrolidone). The coating can exhibit sealing capabilities at least when exposed to the liquid such that, if the coating is in contact with at least one other contact surface, a passage of, e.g., a liquid is prevented.

By providing the connection element with a hydrophilic coating, the connection element can be moved/slide to a desired target location without being affected by friction, also, if the swellable liquid has been exposed to a liquid. Therefore, a single coating can provide two advantageous effects: sealing capabilities while still supporting movability of the connection element.

The rolling membrane can further include at its proximal portion a sealing element, preferably a hemostatic valve.

By providing the rolling membrane with the hemostatic valve at its proximal portion, a secure and sterile insertion of medical instruments into an inner lumen of the rolling membrane can be facilitated. Additionally, a fluid supply to the rolling membrane can be facilitated allowing an inflation of the rolling membrane.

The catheter can further include a manipulation element for manipulating the sealing element. The manipulation element can be insertable into the inner lumen of the rolling membrane from a proximal direction.

The manipulation element can be adapted for pushing the sealing element in the distal direction (from a proximal direction). Additionally or alternatively, the manipulation element can be adapted to pull the sealing element in the proximal direction (from the proximal direction). Additionally or alternatively, the manipulation element can be adapted to be rotatable about its longitudinal axis, e.g. such as to rotate the sealing element.

The manipulation element can allow to manipulate the sealing element from a remote location (e.g., from outside the body of the patient) which can contribute to a simplified manipulation of the catheter, in particular, when inserted into the body of the patient.

The sealing element can be adaptable in diameter by the manipulation element.

The sealing element can be provided with a variable diameter. The variable diameter of the sealing element can mechanically be manipulated. The sealing element can be remotely manipulated, from outside the body of a patient.

By providing the sealing element in an adaptable manner, the sealing element can easily be adapted to the situational needs. By way of example, the sealing element can be set to a small diameter when the sealing element is to be inserted into, e.g., the outer shaft such that the sealing element does not unintentionally counteract an insertion. However, when the sealing element has successfully been inserted into, e.g., the outer shaft, and pushed to a desired target location, the diameter of the sealing element can be increased such that desired sealing properties are exhibited.

The rolling membrane can be adapted for forming an inner lumen, at least when the rolling membrane is in a rolled-out state.

If the rolling membrane is in an at least partially rolled-in state, the inner lumen of the rolling membrane can effectively be occupied by the rolling membrane folded therein. In some exemplary implementations, the inner shaft can not enter the inner lumen of the rolling membrane when the rolling membrane is in a rolled-out state.

A formation of an inner lumen in the rolling membrane can allow a delivery of a medical device (e.g., another catheter and/or a stent, etc.) and/or of a medical drug to a target location which can preferably be located distal to a distal-most tip of the rolling membrane. The delivery can preferably be based on inserting the device and/or the drug to be delivered into the inner lumen of the rolling membrane from a proximal direction of the rolling membrane and a pushing of the device and/or the drug in a distal direction, as seen when the rolling membrane is fully rolled out.

The connection element can be adapted for pressure tightly sealing an inflatable volume of the rolling membrane.

A pressure tight sealing can be understood as a sealing which can not allow any penetration of fluid particles from, e.g., a volume located proximal to the connection element to a volume that lies distal to the connection element up to a certain threshold pressure.

If the connection between the rolling membrane and the outer shaft is adapted to be pressure tight and/or fluid tight, the rolling membrane can be inflatable, e.g., from a proximal direction of the connection element, e.g., by supplying a pressurized liquid and/or a gas. By the connection element, the rolling membrane can in particular be inflatable if the rolling membrane is combined with an outer shaft.

A second aspect of the present invention relates to a method for using a guide extension catheter, preferably the catheter as described above. The method can include providing an outer shaft and providing a rolling membrane connected to an inner shaft at a first portion of the rolling membrane. The method can further include releasably/temporarily connecting a second portion of the rolling membrane to the outer shaft, preferably via a connection element, such that the rolling membrane can be rolled out along a longitudinal axis of the outer shaft.

By the aforementioned method, the catheter can more simply be adapted to situational needs, even when partially be inserted into a blood vessel of a patient.

The method can further include inserting a distal end of the outer shaft into a blood vessel and at least partially rolling out a distal end of the rolling membrane along the blood vessel such that an inner lumen can be formed in the rolling membrane for facilitating a delivery of a medical device and/or a medical drug from a proximal end of the rolling membrane to a distal end of the rolling membrane.

By operating the catheter according to the aforementioned method, the synergetic effects of an extended catheter and a rolling membrane can advantageously be used. This can improve the user experience for a member of a medical staff and can reduce the risk for the patient, e.g., arising from a movement of the catheter towards a distal direction.

A third aspect of the present invention relates to a guide extension catheter which includes or consists of a rolling membrane, a proximal shaft and an outer shaft, preferably a guiding catheter. The proximal shaft can be considered as an inner shaft which is partially arranged within the outer shaft, preferably the guiding catheter. The proximal shaft can include an inner lumen.

The rolling membrane includes a first portion and a second portion. The first portion can be arranged at a distal end of the rolling membrane whereas the second portion can be arranged at a proximal end of the rolling membrane. The rolling membrane can be configured to form an inner rolling membrane lumen in a rolled-in state and/or in a rolled-out state of the rolling membrane. This inner rolling membrane lumen has a larger diameter than conventional inner shafts. Thus, the diameter of the inner rolling membrane lumen enables for guiding (medical and/or sensoring) devices through the inner rolling membrane lumen (e.g. whenever the rolling membrane function is not required).

The proximal shaft can be connected to the second portion of the rolling membrane. Preferably, the proximal shaft can be connected to the rolling membrane such that the proximal shaft at least partially accommodates the second end of the rolling membrane in the inner lumen of the proximal shaft. In other words, the rolling membrane can be connected to an inner side of the proximal shaft. As a result, the proximal shaft can not limit the inner diameter of an inner rolling membrane lumen. The proximal shaft can have a push-pull function, namely to push the rolling membrane in a distal direction (towards a patient) and pull the rolling membrane in a proximal direction (towards the operator/doctor).

The outer shaft, preferably the guiding catheter, can be adapted to at least partially enter a body of a patient with a portion I of the outer shaft. The outer shaft, preferably the guiding catheter, can further be adapted to at least partially remain outside the body of the patient with a portion of the outer shaft.

A diameter of the outer shaft, preferably the guiding catheter, can increase at its proximal part (at the portion O which stays outside the body) compared to its distal part. The diameter of the guiding catheter can increase at its proximal part, such that the proximal shaft can not enter a distal part of the guiding catheter. Because the outer shaft, preferably the guiding catheter, has a smaller diameter at its distal portion than at its proximal portion, it hinders the proximal shaft to be inserted into the distal portion of the outer shaft. Therefore, the length of the rolling membrane to exit the outer shaft, preferably the guiding catheter, in the rolled out state can be defined by the length of the distal portion of the outer shaft, preferably the guiding catheter, having a smaller diameter than the proximal portion of the outer shaft. The proximal shaft can be adapted to remain outside of a body of a patient during use of the catheter with the patient. In other words, the proximal shaft can be adapted to not enter the body of a patient.

The rolling membrane can be adapted to be rolled out along a distal direction. The rolling membrane can be adapted to be rolled in along a proximal direction. The rolling membrane can be configured such that the rolling membrane extends to a larger extent in the longitudinal direction than it extends along a radial direction. In some embodiments, the first portion of the rolling membrane can be a distal end of the rolling membrane as seen if the rolling membrane is fully rolled out. Thus, the rolling membrane is configured to exit the outer shaft, preferably the guiding catheter, in the distal direction.

The outer shaft, preferably the guiding catheter, accommodates at least a portion of the rolling membrane. At least a distal portion of the outer shaft, preferably the guiding catheter, can be connected to the first portion of the rolling membrane.

The proximal shaft can be adapted to push the rolling membrane into the distal direction D and/or to pull the rolling membrane in a proximal direction P. The inner lumen of the proximal shaft can be connected to the inner lumen of the rolling membrane such that, e.g., medical devices and/or medical drugs can be fed from a proximal portion of the catheter to a distal portion of the catheter.

The rolling membrane can have a smaller wall thickness than the inner shaft. Therefore, it compromises the inner rolling membrane lumen less than a conventional inner shaft.

The proximal end of the rolling membrane can be connected to an inner side of the proximal shaft and wherein a distal end of the rolling membrane can be connected to outer shaft, preferably the guiding catheter. The outer shaft can accommodate at least a portion of the rolling membrane.

At a proximal end portion of the outer shaft, preferably the guiding catheter, the outer shaft can be terminated with a sealing element. The sealing element can preferably be a hemostatic valve. The proximal shaft can at least partially extend through the sealing element.

A fourth aspect of the present invention relates to a guide extension catheter which includes or consists of a rolling membrane, an outer shaft (e.g. a guiding catheter) through which the rolling membrane partially extends through, and wherein a proximal portion of the rolling membrane is slidably connected to a sealing valve and a distal portion of the rolling membrane is connected to a connection element.

The sealing valve and/or the connection element can include a hydrophilic coating and/or a swellable coating such as to swell if exposed to a liquid. The sealing valve can include at least one hemostatic valve, for example the sealing valve can be a Tuohy Borst valve.

The sealing valve can be limited in its distal movement by the outer catheter. For example a distal end of the sealing valve and a proximal end of the outer catheter can form a positive connection (form fit connection). For connecting the outer shaft to the valve 611, the valve can include a shaft shaped portion, directed in the distal direction, and including and outer diameter slightly smaller in diameter than an inner diameter of the outer shaft. The shaft shaped portion can be provided with an inner diameter of at least 4 Fr.

The outer shaft can be movably arranged on the rolling membrane. The outer shaft can be moved in a proximal direction, wherein, as a result of the translation of the outer shaft, the connection element can (further) enter the outer shaft in a distal direction. Said sliding movement of the connection element can advantageously be supported by a preferred hydrophilic coating of the connection element which can reduce friction during the sliding translation.

The rolling membrane can extend through the sealing valve (e.g. Tuohy Borst valve). In some embodiments, the rolling membrane can be connected to a proximal shaft in a proximal end portion of the rolling membrane.

The connection element can be adapted to seal a distal end portion of the catheter.

The guide extension catheter according to the present invention can synergistically combine the advantageous of a guide extension catheter (as described above) and a rolling membrane for improving catheter-based inventions as described above.

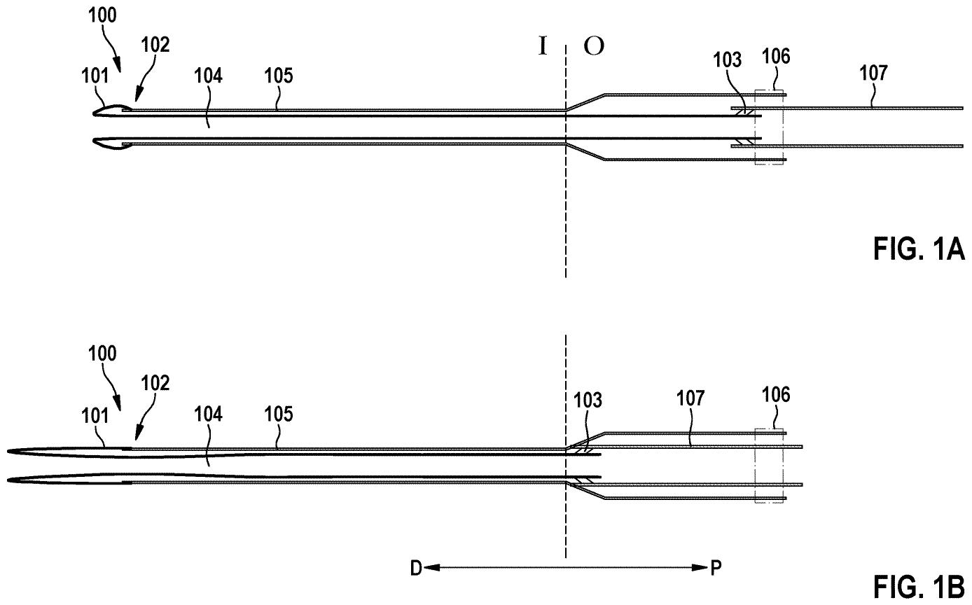

FIGS. 1A and 1B depict a first exemplary embodiment of a rolling membrane catheter according to an aspect of the present invention.

FIG. 1A shows an exemplary embodiment of a catheter 100 which includes a rolling membrane 101 (depicted in a rolled-in state). The rolling membrane 101 includes a first portion 102 and a second portion 103. The first portion 102 can be arranged in a distal portion of the rolling membrane 101 whereas the second portion 103 can be arranged in a proximal portion of the rolling membrane 101. The rolling membrane 101 can be configured to form an inner lumen 104 in a rolled-in state of the rolling membrane 101 and/or in a rolled-out state of the rolling membrane 101.

The catheter 100 can further include an outer shaft 105, which can accommodate at least a portion of the rolling membrane 101. The outer shaft 105 can be a guiding catheter. The outer shaft 105 can be adapted to at least partially enter a body of a patient with a portion I of the outer shaft 105. The outer shaft 105 can further be adapted to at least partially remain outside the body of the patient with a portion O of the outer shaft 105. A diameter of the outer shaft 105 can increase at its proximal part (at the portion O which stays outside the body) compared to its distal part.

At least a distal portion of the outer shaft 105 can be connected to the first portion 102 of the rolling membrane 101. At a proximal end portion of the outer shaft 105, the outer shaft 105 can be terminated with a sealing element 106. The sealing element 106 can preferably be a hemostatic valve.

The catheter 100 can further include a proximal shaft 107. The proximal shaft 107 can at least partially extend through the sealing element 106. The proximal shaft 107 can include an inner lumen. The proximal shaft 107 can be connected to the second portion 103 of the rolling membrane 101. Preferably, the proximal shaft 107 can be connected to the rolling membrane 101 such that the proximal shaft 107 at least partially accommodates the second end 103 of the rolling membrane 101 in the inner lumen of the proximal shaft 107. In other words, the rolling membrane 101 can be connected to an inner side of the proximal shaft 107. As a result, the proximal shaft 107 can not limit an inner diameter of the inner lumen 104 of the rolling membrane 101 and therefore of the outer shaft (guiding catheter) 105 itself.

The proximal shaft 107 can be adapted to always remain outside the body of the patient, preferably in a portion O. The proximal shaft 107 can be adapted to push the rolling membrane 101 into the distal direction D and/or to pull the rolling membrane 2 in a proximal direction P. The inner lumen of the proximal shaft 107 can be connected to the inner lumen 104 of the rolling membrane 101 such that, e.g., medical devices and/or medical drugs can be fed from a proximal portion of the catheter 100 to a distal portion of the catheter 100.

FIG. 1B shows the exemplary implementation of a catheter 100 of FIG. 1A, wherein the rolling membrane 101 has fully been rolled out along the distal direction D. The proximal shaft 107 can remain within the outer shaft 105 and particular that portion of outer shaft 105 that is outside the patient and can have an increased diameter.

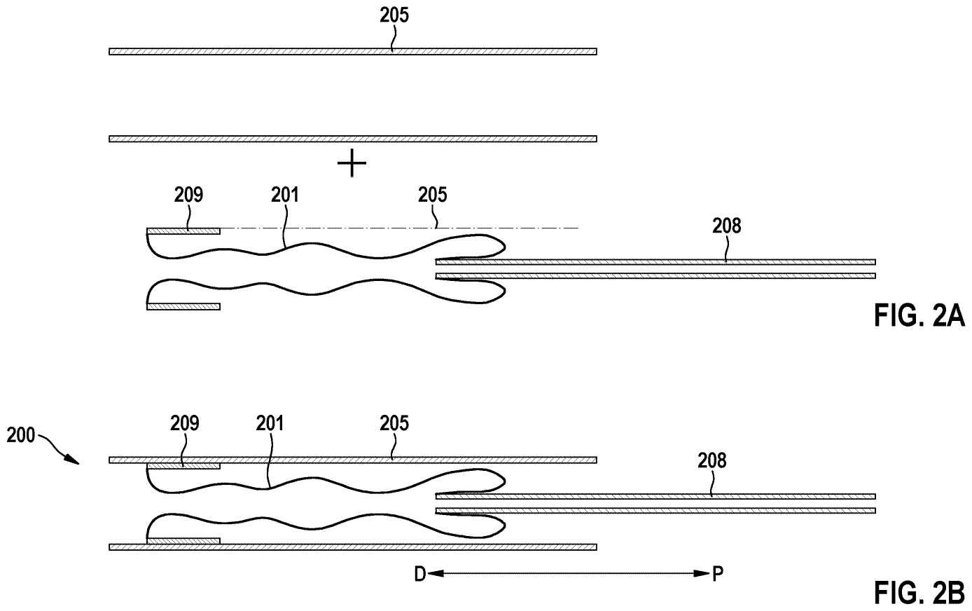

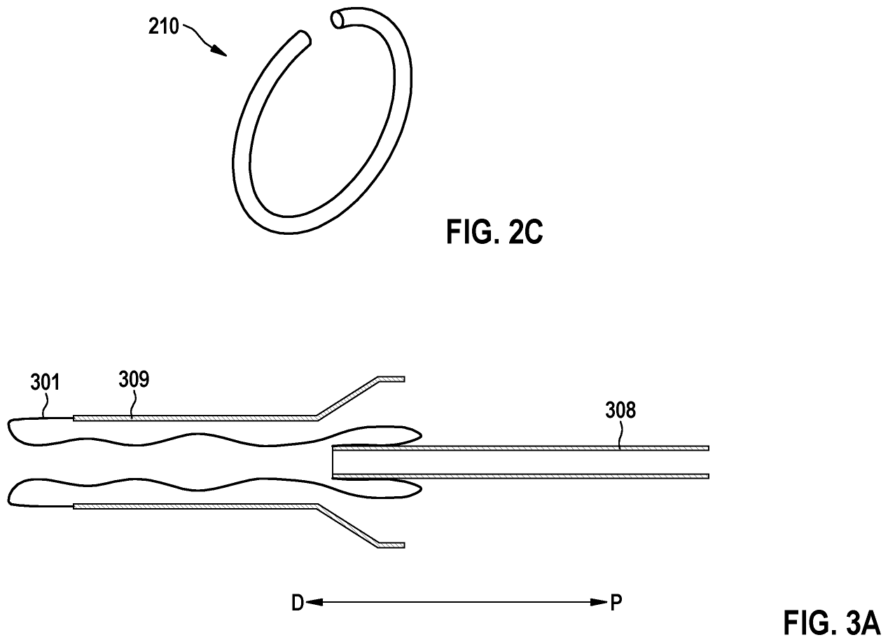

FIGS. 2A-B shows a further embodiment of a catheter 200 according to an aspect of the present invention.

Notably, FIG. 2A illustrates the combination of an outer shaft 205 with a rolling membrane 201 for forming a rolling membrane guide extension catheter (RMGCE). The rolling membrane 201 can be provided with a length between 1 cm and 30 cm (as seen in a fully rolled-out state of the rolling membrane 201).

Rolling membrane 201 can be connected to an inner shaft 208. Rolling membrane 201 can be connected to the inner shaft 208 in a distal portion of inner shaft 208. Rolling membrane 201 can be connected to the inner shaft 208 at an outer side of the inner shaft 208. Rolling membrane 201 can be connected to the inner shaft 208 by welding, gluing, clamping and/or any other suitable method.

The catheter can further include a connection element 209. Connection element 209 can be provided as an annular element, e.g., a ring such as a circlip 210 as exemplarily depicted in FIG. 2C. In some exemplary implementations, the connection element 209 can be implemented as an elastic stent-like structure. In some exemplary implementations, the connection element 209 can be provided as a spring seal element.

The connection element 209 can be placed at some distance to the distal end of the outer shaft 205 to allow the rolling membrane 201 to be self-sealing against the inner side of the outer shaft 205. If the connection element 209 is provided as the spring seal element, the spring seal element must assure a sufficient pressure loss for any liquid passing between spring element and the outer shaft 205 (e.g., a guiding catheter), so that the pressure within an inflatable volume of the rolling membrane 201 is higher than on the outside of the rolling membrane 201.

The connection element 209 can preferably be adapted to be slidable in the inner lumen of the outer shaft 205 and then fixed therein. The connection element 209 can alternatively also be adapted to be put over the outer side of the outer shaft 205 and clamp the rolling membrane 201 onto the outer shaft 205.

In an exemplary implementation of the catheter 200, when the rolling membrane 201 is fully rolled out, the portion of the rolling membrane 201 in contact with connection element 209 can form the proximal end of the rolling membrane. Inner shaft 208 can bring the portion of the rolling membrane 201 connected to the inner shaft 208 to a distal-most position.

FIG. 2B shows the outer shaft 205 and connection element 209 connected, wherein the connection element 209 is adapted to be inserted into the inner lumen of the outer shaft 205. The connection element is connected to a distal part of outer shaft.

The connection element 209 can be provided as an annular element (as described with reference to FIG. 2A, above) and can be provided with a diameter which can be larger than an inner diameter of the outer shaft 205. When inserting the connection element 209 into the inner lumen of the outer shaft 205, the connection element 209 can exhibit a force onto the rolling membrane 201 and the inner side of the outer shaft 205 such that the rolling membrane 201 is at least temporarily connected to the outer shaft 205.

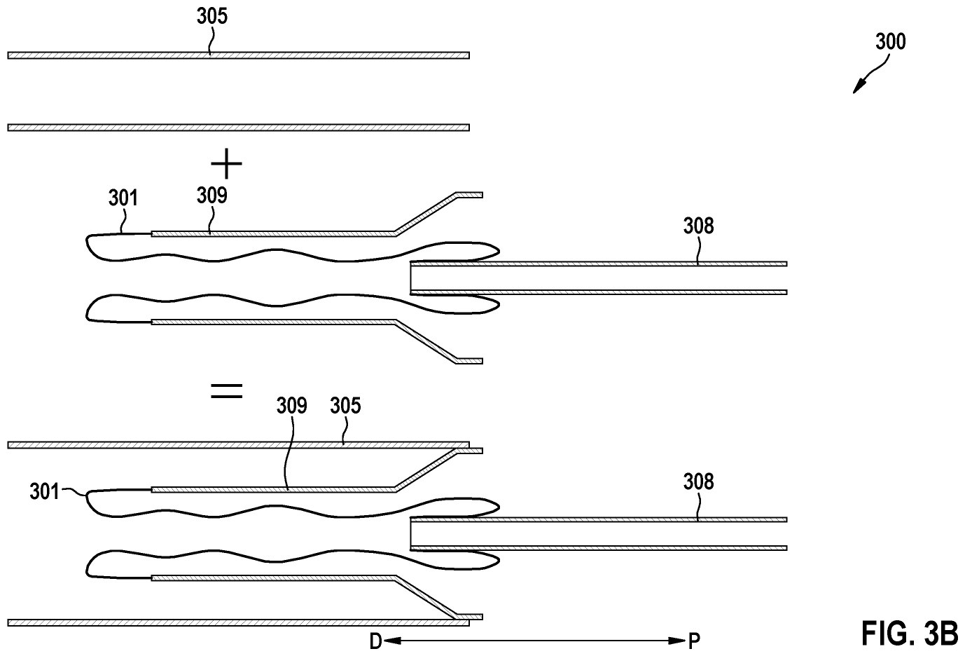

FIGS. 3A and 3B depict a further exemplary embodiment of a connection element 309. The connection element 309 is attached to a distal portion of a rolling membrane 301 and a proximal portion of the rolling membrane is attached to an inner shaft 308 preferably at the proximal side.

FIG. 3A shows an exemplary illustration of the connection element 309 according to a further aspect of the present invention. In this exemplary implementation, the connection element 309 can be provided as funnel-shaped element, which can include a first diameter at a proximal portion of the connection element 309 which can be smaller or equal (at a rest state, i.e., a state in which the connection element 309 is not connected to an outer shaft 305) than the inner diameter of the outer shaft 305.

FIG. 3B shows an exemplary combination of the outer shaft 305, which can be configured as described, above, and the connection element 309. The connection element 309 can be insertable into the inner lumen of the outer shaft 305. The connection element is connected to a proximal part of outer shaft. The connection element 309 can be connected to the inner side of the outer shaft 305 and can be locked in the inner lumen of the outer shaft 305 by the proximal portion of the connection element 309 which can be compressed in diameter and thus radial press against the inner sides of outer shaft 305 at its proximal side. Thereby, a temporary connection of the connection element 309 and the outer shaft 305 can be facilitated.

With respect to the proximal portion of the connection element 309, the diameter of the connection element 309 can generally be provided with a diameter which can allow an insertion of the connection element 309 into the outer shaft 305. By way of example, if the rolling membrane 301 is provided as a 4 Fr element and if the outer shaft is a 6 Fr element, the proximal portion of the connection element 309 can be provided with 6 Fr or less to create a sealing between outer shaft 305 and connection element 309.

Preferably, the outer shaft 305 can be provided with an inner diameter that is 1 Fr larger than the diameter of the rolling membrane 301 for facilitating an application of a mother-child technique (e.g., a smaller catheter can be inserted into the inner volume of a bigger catheter). In a most preferred embodiment, the outer shaft 305 can be provided as a 6 Fr part and the connection element can be provided as a 4 Fr part.

By inserting the connection element 309, as depicted in FIGS. 3A and 3B, into the outer shaft 305, a rolling membrane guide extension catheter (RMGCE) can be formed by a member of a medical staff. In a preferred embodiment, the RMGCE can be formed from any guiding catheter acting as the outer shaft 305.

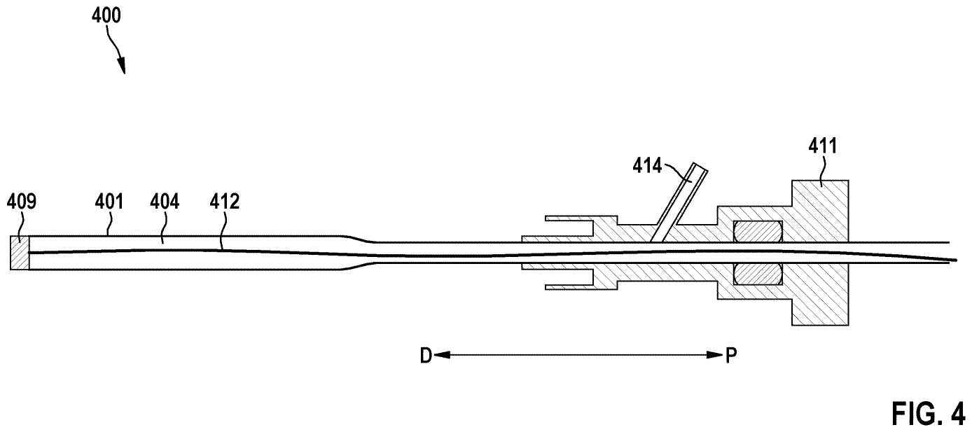

FIG. 4 depicts a further exemplary embodiment of a catheter 400, notably of a rolling membrane catheter which can include an inner shaft (not shown) with an additional tension lock for forming a rolling membrane guide extension catheter (RMGCE) which needs to be inserted into a guiding catheter of the right inner diameter. The catheter 400 can include a rolling membrane 401 extending axially from a distal direction D to a proximal direction P.

At a proximal end of the rolling membrane 401 (as seen if the rolling membrane 400 is fully rolled out), the rolling membrane 401 can be connected to sealing and/or connecting element 409. Sealing element 409 can include a hydrophilic coating and/or a swellable coating. Sealing element 409 can preferably include a sealing element. The sealing element can be provided as a spring sealing element.

The rolling membrane 401 can be passed through a valve 411, e.g., a Tuohy borst adapter. The valve 411 can preferably be placed outside of a body of a patient. Valve 411 can be provided with means 414 for providing a fluid (e.g., a liquid and/or a gas) to an inflatable volume of the rolling membrane 401.

Catheter 400 can further include a manipulation element 412 arranged in an inner lumen 404 of the rolling membrane 401. The manipulation element 412 can be connected to the sealing element 409 and can extend or be operably coupled to a position outside the patient. The manipulation element 412 can be adapted for avoiding that the sealing element 409 (and the rolling membrane 401) unintentionally enters the blood vessel of the patient by a too large distance (in particular, if the rolling membrane 401 is inflated the hydraulic pressure applied to the rolling membrane 401 will pull the sealing element 409 out of the guiding catheter). Manipulation element 412 can allow to place the sealing element 409, e.g. within an outer shaft (not shown)

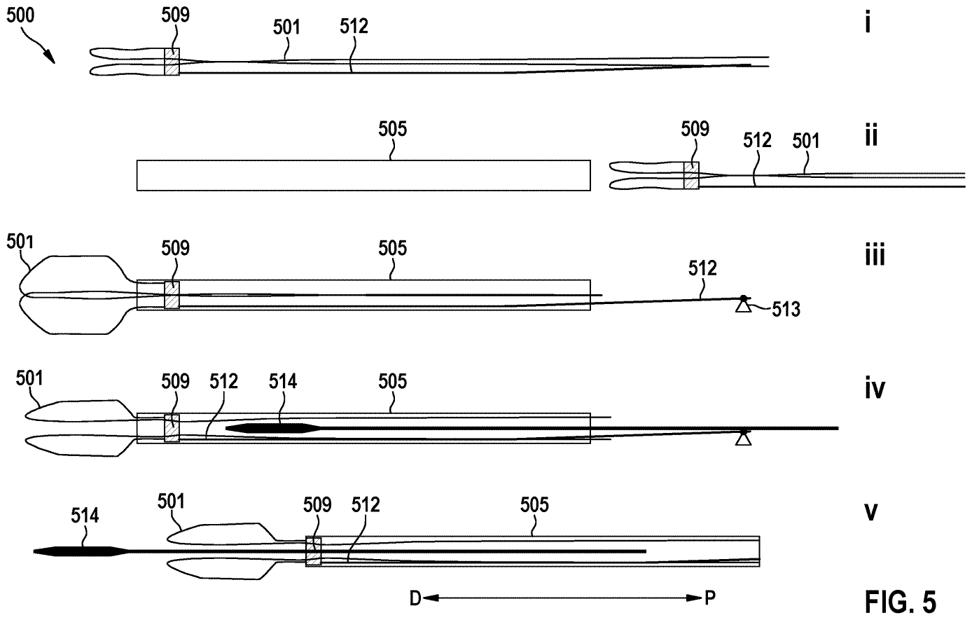

FIG. 5 exemplarily illustrates an insertion of rolling membrane 501 (e.g., the rolling membrane 401 as described with reference to FIG. 4, above) into an outer shaft 505 (e.g., a guide catheter such as to form an RMGCE) in five stages i)-v).
Stage i) shows the rolling membrane 501 which can be configured as described with reference to FIG. 4, above.
Stage ii) shows the rolling membrane 501 to be inserted into the outer shaft 505. The maximum radius of the rolling membrane 501 can be configured to be smaller than an inner diameter of the outer shaft 505.
Stage iii) shows a scenario in which the rolling membrane 501 is inserted into the outer shaft 505. Connection and/or sealing element 509 (preferably provided with a hydrophilic and swellable coating) can have been exposed to a liquid such that a sealing of the inner lumen of the outer shaft 505 can be exhibited. The sealing can cause a preferably gas tight separation from a distal volume portion of the inner lumen of the outer shaft 505 from a proximal volume portion of the inner lumen of the outer shaft 505. It can releasably connect the rolling membrane 501 to the outer shaft 505 (e.g. a proximal portion of the rolling membrane).
Stage iii) further depicts a blocking element 513 which can include a volume element which is configured with dimensions that exceed a diameter of the outer shaft 505 such that the blocking element can not be able to enter the outer shaft 505, e.g., from a proximal direction P. This configuration can ensure that the manipulation element 512 can not accidentally enter a blood vessel of a patient.
Stage iii) further depicts the rolling membrane 501 in an at least partially rolled-out state. Stage iv) depicts the rolling membrane 501 of stage iii) wherein a medical device 514 has been inserted into an inner lumen 504 of the rolling membrane 501. The medical device 514 may, e.g., be a catheter which can be inserted from the proximal direction P into the distal direction D through the inner lumen of the rolling membrane 501.
Stage v) depicts the rolling membrane 501 of stage iv) wherein the medical device 514 has been delivered to a position which lies distal to a distal-most tip of the rolling membrane 501.

Generally, the rolling membrane 501 can preferably be everted into a narrow, tortuous blood vessel. Upon everting the rolling membrane 501 into the blood vessel (as shown in stage iii), the rolling membrane 501 can be at least partially deflated (stage iv) and any catheter smaller than 4 Fr can be pushed towards the target location.

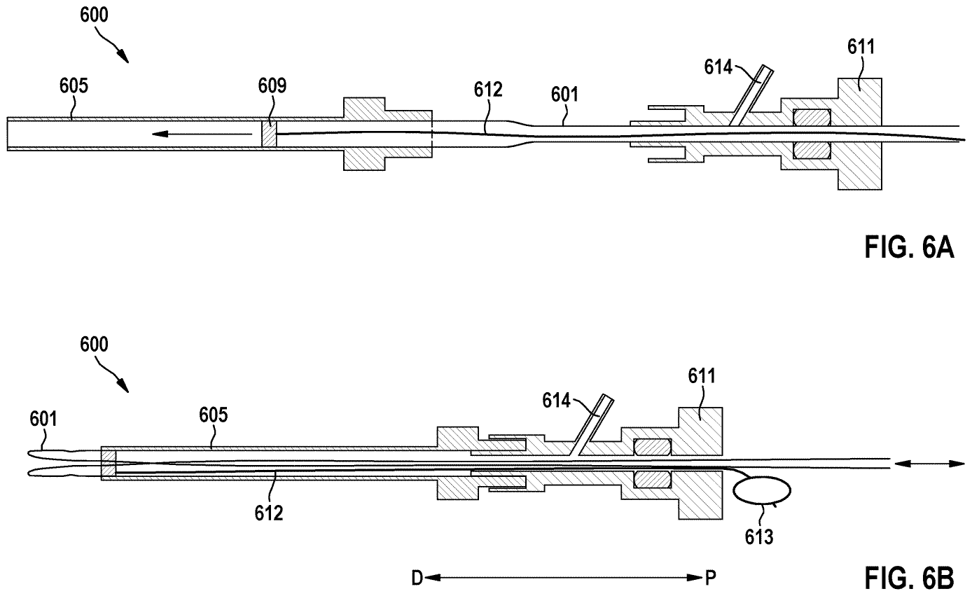

FIGS. 6A and 6B exemplarily show a catheter 600 as it has been described with reference to FIGS. 4 and 5, above.

FIG. 6A shows a potential combination of a rolling membrane 601 with an outer shaft 605 (e.g., the outer shaft 505 as described with reference to FIG. 5, above) and a valve 611 (e.g., valve 411 as described with reference to FIG. 4, above). In FIG. 6A, the rolling membrane 601 has at least partially been inserted into the outer shaft 605. Outer shaft 605 can be moved in a proximal direction P, wherein, as a result of the translation of the outer shaft 605, the connection element 609 can further enter the outer shaft 605 in a distal direction. Said sliding movement of the connection element 609 can advantageously be supported by a preferred hydrophilic coating of the connection element 609 which can reduce friction during the sliding translation.

FIG. 6B shows the catheter of FIG. 6A, wherein the outer shaft 605 has been contacted with the valve 611 by moving the outer shaft 605 of FIG. 6A further in the proximal direction thus forming an RMGCE.

FIG. 6B further shows the rolling membrane 601 in an at least partially everted state.

In a preferred embodiment, the rolling membrane 601 can include a diameter that is smaller than a diameter of a blood vessel of a patient. Moreover, the rolling membrane 601 can include a diameter that can be larger in diameter than an inner diameter of the outer shaft 605 (at least in a rolled-out state of the rolling membrane 601).

In some embodiments, the rolling membrane 601 can be connected to a proximal shaft in a proximal end portion of the rolling membrane 601.

Connection element 609 can further be adapted to seal the catheter 600 in distal end portion of the catheter 600.

For connecting the outer shaft 605 to the valve 611, the valve 611 can include a shaft shaped portion, directed in the distal direction D, and including and outer diameter slightly smaller in diameter than an inner diameter of the outer shaft 605. The shaft shaped portion can be provided with an inner diameter of at least 4 Fr.

In some exemplary embodiments, the catheter 600 can be provided with an additional tube inside the inner lumen of the rolling membrane 601 as an additional manipulation element 612 for manipulating the connection element 609. Valve 611 can be configured to be pressure tight and can be adapted to seal a proximal end portion of the catheter 600.

Volume element 613 can be similar to volume element 513 outlined with reference to FIG. 5.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A guide extension catheter comprising:
a rolling membrane;
an inner shaft;
wherein a first portion of the rolling membrane is connected to the inner shaft;
a connection element configured to temporarily connect a second portion of the rolling membrane to an outer shaft.

2. The guide extension catheter of claim 1, wherein the connection element is connected to a proximal portion of the rolling membrane.

3. The guide extension catheter of claim 1, wherein the connection element is configured to be inserted into the outer shaft to establish a clamping connection between the rolling membrane and the outer shaft.

4. The guide extension catheter of claim 1, wherein the connection element comprises a first diameter at a proximal portion of the connection element, the first diameter being equal or smaller than an inner diameter of the outer shaft, wherein the connection element comprises a second diameter at a distal portion of the connection element, is the second diameter being smaller than the first diameter.

5. The guide extension catheter of claim 1, wherein the connection element is connectable to a distal portion of the rolling membrane.

6. The guide extension catheter of claim 5, wherein the connection element comprises an annular element for exerting a radially acting force onto a distal portion of the rolling membrane and an outer side of the outer shaft, or an inner side of the outer shaft.

7. The guide extension catheter of claim 5, wherein the connection element is configured to be put over the outer shaft and to establish a clamping connection between the rolling membrane and the outer shaft.

8. The guide extension catheter claim 5, wherein the connection element comprises a hydrophilic coating and/or a swellable coating.

9. The guide extension catheter of claim 1, wherein the rolling membrane comprises at its proximal portion a sealing element.

10. The guide extension catheter of claim 9, comprising:
a manipulation element configured to manipulate the sealing element, the manipulation element being insertable into an inner lumen of the rolling membrane from a proximal direction.

11. The guide extension catheter of claim 10, wherein the manipulation element is configured to change a diameter of the sealing element.

12. The guide extension catheter of claim 1, wherein the rolling membrane is configured to form an inner lumen when the rolling membrane is in a rolled-out state.

13. The guide extension catheter of claim 1 wherein the connection element is configured to tightly seal an inflatable volume of the rolling membrane.

14. A method for using a catheter comprising:
providing an outer shaft;
providing a rolling membrane connected to an inner shaft at a first portion of the rolling membrane;
releasably connecting a second portion of the rolling membrane to the outer shaft via a connection element such that the rolling membrane can be rolled out along a longitudinal axis of the outer shaft.

15. A guide extension catheter comprising:
a rolling membrane;
a proximal shaft; and
a guiding catheter,
wherein a proximal end of the rolling membrane is connected to an inner side of the proximal shaft and wherein a distal end of the rolling membrane is connected to the guiding catheter.

16. The guide extension catheter according to claim 15, wherein a diameter of the guiding catheter increases at its proximal part, such that the proximal shaft cannot enter a distal part of the guiding catheter.

17. The guide extension catheter according to claim 15, wherein a proximal end portion of the guiding catheter is connected to a sealing element.

18. The guide extension catheter according to claim 17, wherein the sealing element is a hemostatic valve.

19. The guide extension catheter according to claim 17, wherein the proximal shaft at least partially extends through the sealing element.

20. A guide extension catheter comprising:
a rolling membrane; and
an outer shaft through which the rolling membrane partially extends through, wherein a proximal portion of the rolling membrane is slidably connected to a sealing valve and a distal portion of the rolling membrane is connected to a connection element.

21. The guide extension catheter according to claim 20, wherein the outer shaft is a guiding catheter.

22. The guide extension catheter according to claim 20, wherein the sealing valve and/or the connection element comprises a hydrophilic coating and/or a swellable coating.

23. The guide extension catheter according to claim 20, wherein the sealing valve comprises at least one hemostatic valve.

24. The guide extension catheter according to claim 20, wherein the sealing valve is a Tuohy Borst valve.

25. The guide extension catheter according to claim 20, wherein the rolling membrane extends through the sealing valve.

26. The guide extension catheter according to claim 20, wherein the sealing valve is limited in its distal movement by the outer catheter.

27. The guide extension catheter according to claim 20, wherein the connection element is configured to seal a distal end portion of the catheter.

28. The guide extension catheter according to claim 20, wherein a distal end of the sealing valve and a proximal end of the outer catheter are configured to form a positive connection.
